(19) United States
(12) Patent Application Publication
(10) Pub. No.: US 2014/0096284 A1
(43) Pub. Date: Apr. 3, 2014

(54) METHOD FOR THE DELIVERY OF MOLECULES LYOPHILIZED ONTO MICROPARTICLES TO PLANT TISSUES

(71) Applicant: Iowa State University Research Foundation, Inc., Ames, IA (US)

(72) Inventors: Susana Martin-Ortigosa, Ames, IA (US); Kan Wang, Ames, IA (US)

(21) Appl. No.: 13/785,433

(22) Filed: Mar. 5, 2013

Related U.S. Application Data
(60) Provisional application No. 61/708,329, filed on Oct. 1, 2012.

(51) Int. Cl. C12N 15/82; C12N 15/63; A61K 9/50

(52) U.S. Cl. CPC C12N 15/8207; A61K 9/5089; C12N 15/63
USPC 800/293; 424/490; 424/499; 435/470; 435/410; 424/184.1; 424/204.1; 424/234.1; 424/275.1; 435/459; 514/44 R; 514/1.1; 435/375

(57) ABSTRACT

The invention provides particles and methods to deliver freeze- or air-dried molecules to cells.

### CROSS-REFERENCE TO RELATED APPLICATIONS

This application claims the benefit of the filing date of U.S. application Ser. No. 61/708,329, filed on Oct. 1, 2012, the disclosure of which is incorporated by reference herein.

### BACKGROUND

Studying the role of the components found in living cells is one of the major purposes of basic biology research. These components are biomolecules or chemical products that have a particular function of interest in the cell. A traditional way of studying the function of such molecules has been the manipulation of the genome, by the over-expression or silencing of nucleic acid sequences like genes or regulatory sequences related to the molecule of interest.

In recent years, delivery of molecules other than nucleic acids has been a major focus of study, mostly in biomedical sciences, where different drug delivery systems have been developed (Ravichandran, 2009). The intracellular delivery of chemicals (e.g., hormones, dyes or inhibitors), proteins (e.g., antibodies, labeled proteins or enzymes) and other biomolecules or a combination of molecules has an enormous potential for basic and applied research. The direct intracellular delivery of a protein could, for instance, save the time, cost and labor needed to create a transgenic organism that expresses it. Moreover, the protein could be labeled for monitoring or have different modifications that cannot be achieved by its expression in the host organism. Also, the functional analysis of the protein could be multidimensional if it could be co-delivered with other biomolecules or chemicals related to its role in the cell. For biotechnological purposes, the delivery of enzymes with a specific function for gene editing or DNA recombination could for instance improve plant transformation efficiency or facilitate genome edition.

Several technology platforms have been developed to achieve intracellular protein delivery, like cell penetrating peptides or protein transduction domains (Eggenberger et al., 2009). These short peptides, after incubation with the molecule of interest, are able to cross the cell membrane and deliver the cargo (Eggenberger et al., 2009). This technology has been effective to deliver biomolecules to plant tissues overcoming the plant cell wall (Chugh et al., 2009; Lu et al., 2010; Eggenberger et al., 2011; Qi et al., 2011). However, this technique requires peptide synthesis, incubation parameters optimization, and generally a covalent bond to a protein of interest (Lu et al., 2010), and the uptake could depend on the type of plant tissues (Qi et al., 2011).

Another technology platform that is nowadays a major focus of research is nanoparticle mediated drug delivery (Ravichandran, 2009). Most of the research in this area is in the biomedical field, where nanoparticles are being tailored to deliver a particular molecule to a specific cell under certain conditions. Nevertheless, nanoparticle mediated molecule delivery to plant cells has been reported, including delivery of nucleic acids (Vijayakumar et al., 2010; Wang et al., 2011; Martin-Ortigosa et al., 2012a; Martin-Ortigosa et al., 2012b; Naqvi et al., 2012), proteins (Martin-Ortigosa et al., 2012a), and chemicals (Grichko et al., 2006; Torney et al., 2007; Wild and Jones, 2009). The suitability of mesoporous silica nanoparticles for chemical and DNA (Torney et al., 2007), or protein and DNA co-delivery (Martin-Ortigosa et al., 2012a) through the biolistic method, has also been reported. The use of nanoparticles for delivery of substances to plant cells is a technology currently in development and it can require nanoparticle modifications such as increasing nanoparticle density for a better biolistic delivery to plant tissues (Martin-Ortigosa et al., 2012b), or increasing the pore size for protein loading (Martin-Ortigosa et al., 2012a).

In order to improve plant genetic transformation, Wu and colleagues followed two biolistic approaches to deliver transposomes using 1 μm gold microparticles (Wu et al., 2011a; Wu et al., 2011b). In both cases, the surface of the gold microparticle had to be modified to promote the binding of helper proteins that locked the transposome to the projectile and favored the release of that complex once it was intracellularly delivered. Also, the transposon sequence or the transposase itself had to be engineered to form this microparticle-protein-DNA complex (Wu et al., 2011a; Wu et al., 2011b).

More than a decade ago the term “diolistics” was used for the first time to refer to a technique in which dyes were dried onto microprojectiles and bombarded to nervous system cells for labeling (Gan et al., 2000). This technique has been used to deliver dyes indicators of cellular physiological state or permitted the visualization of cell architecture (O'Brien and Lummis, 2007; Roizenblatt et al., 2006). This method has also been used in plant and algal cells to monitor changes in cytosolic calcium concentrations (Bothwell et al., 2006). In this technique, a dye (chemical compound) is solubilized in a solvent like water (Roizenblatt et al., 2006) or methylene chloride (Gan et al., 2000). This solution is mixed with a particle suspension (gold or tungsten particles of 0.6-1.7 μm in diameter). This mix is usually poured over a glass slide and left to dry by evaporation. While the solvent evaporates, the dye is precipitated over the surface of the particles. The dye coated projectiles are then used for bombardment of different tissues. Delivery of RNA to parasitic helminths has also been achieved by the biolistic method, lyophilizing the RNA onto 1.6 μm gold microcarriers (Davis et al., 1999).

### SUMMARY OF THE INVENTION

The present invention provides for the coating of particles, such as microparticles or nanoparticles (collectively “particles”), e.g., those formed of gold, tungsten, mesoporous silicate, silver, quantum dots, carbon nanotubes, or polystyrene beads, with molecules including molecules of biological origin (biomolecules) and chemicals (agents that are not obtained from cells or viruses) using lyophilization (“freeze-drying”) or air-drying (drying that does not rely on sublimation or the use of temperatures below 0° C.; rather it relies on evaporation at temperatures above 0° C., e.g., around 4° C. to about 28° C.). The freeze-dry method uses sublimation to precipitate the molecules over projectiles, thereby preserving the integrity and activity of molecules such as biomolecules that could be degraded or are more sensitive to degradation using simple evaporation methods.

In one embodiment, the present invention employs the biolistic method to deliver, for example, bioactive enzymes, to plant cells using a projectile coating that is achieved by lyophilizing (freeze-drying) or air-drying a solution of proteins along with a projectile suspension. Lyophilization is fast, and prevents macromolecules such as proteins from degradation. Delivery of lyophilized molecules to cells may be useful for transient modifications, e.g., using proteins with a short half-life, or in gene editing, where the delivery of an enzyme involved in DNA recombination, DNA cleavage, or DNA modification could help to engineer accurately plant genomes including organelle genomes such as chloroplasts and mitochondria, thereby improving plant transformation or allowing for precise gene edition. Since the gene gun is also applied in medical sciences for therapeutic purposes, the bombardment of organisms such as humans, or a canine, equine, bovine, swine, caprine, ovine, feline, or non-human primates with lyophilized agents, e.g., proteins or peptides, could deliver vaccines and other therapeutic substances.

As disclosed below, DNA encoding protein or proteins were freeze-dried or air-dried onto the surface of particles and those coated particles were successfully delivered to plant cells and mouse cells after bombardment and the proteins were shown to be active. The present method offers a straight forward, simple and inexpensive way of achieving delivery of any molecule, including proteins or other molecules, and including combinations of molecules, to cells such as plant cells and mammalian cells. The present freeze-dry or air-dry method does not require loading of particle pores with molecules, the use of particle surface modifying moieties, e.g., to covalently link agents to the surface, or techniques for efficient release of the agent once the particles reach the cell.

In one embodiment, solutions of molecules such as proteins, hormones, or enzymes are freeze-dried or air-dried over particles, allowing for layering over the surface of the particles. For instance, a mixture of a solution having one or more isolated molecules and a particle suspension is layered over a macrocarrier (which is also referred to as a cartridge or projectile holder) used for bombardment. This loaded macrocarrier is frozen in liquid nitrogen for several minutes and then freeze-dried in a lyophilizer, or is subjected to air-drying. The methodology works not only with proteins, but also with chemicals and combinations of molecules. The freeze-dry or air-dry coating method thus provides for delivery of molecules such as proteins, or mixtures of molecules, such as plasmid DNA and proteins.

As disclosed herein, particles are not required to deliver molecules using biolistic methods. In one embodiment, a mixture of a solution having one or more molecules is layered over a macrocarrier or other substrate, which is subjected to freeze drying or air drying, and then loaded in the gene gun. Thus, in one embodiment, solutions of molecules such as proteins or nucleic acid are freeze-dried or air-dried, thereby producing a dried materials, which is then layered over a macrocarrier used for bombardment. In another embodiment, a solution of molecules, e.g., liquid droplets having molecules, are placed on over a macrocarrier used for bombardment.

In one embodiment of the invention, the molecule, or particle and molecule containing mixture, can be in cell media, ethanol, water or a buffer, e.g., phosphate buffered saline, prior to freeze-drying or air-drying. Any suitable and effective solvent can be employed.

In one embodiment, the freeze-dry or air-dry process is employed directly with a macrocarrier that is going to be used in bombardment. In another embodiment, the freeze-dry or air-dry process is employed with a solution of particles and at least one agent in a tube, slide or other receptacle, where the coated particles are then loaded in the gene gun.

In one embodiment, the invention provides a plurality of particles such as tungsten or gold particles, e.g., about 0.3 μm to about 3.0 μm in diameter or about 0.2 μm to about 1.2 μm in diameter, having a freeze-dried or air-dried coating of at least one isolated molecule. The molecule may be, for example, isolated protein, isolated hormone, isolated glycoprotein, isolated nucleic acid, e.g., isolated DNA, or a mixture or complex of isolated nucleic acid and isolated protein.

Also provided is a method to prepare particles coated with at least one molecule. The method includes providing a substrate having a solution with a mixture of a plurality of particles, e.g., of about 0.3 μm to about 1.2 μm in diameter, and at least one isolated molecule, and freeze-drying or air-drying the solution in or on the substrate to provide a preparation of particles coated with the at least one molecule. In one embodiment, the molecule is not isolated nucleic acid such as isolated ribonucleic acid. In one embodiment, the at least one molecule is an enzyme.

Further provided is a method to deliver at least one molecule to a eukaryotic cell, such as a plant cell or a mammalian cell. The method includes providing a plurality of particles, for instance, gold particles of about 0.2 μm to about 2 μm in diameter, having a freeze-dried or air-dried coating with at least one isolated molecule; and biolistically delivering the plurality to eukaryotic cells such as mammalian cells in an amount effective to deliver the at least one molecule into the cells. The method also includes providing freeze-dried or air-dried isolated molecules, or a combination of isolated molecules, for instance, about 0.2 μm to about 3 μm in diameter; and biolistically delivering the freeze-dried or air-dried isolated molecules (i.e., in the absence of particles) to eukaryotic cells in an amount effective to deliver the at least one molecule into the cell. In one embodiment, the cells are in a plant. In one embodiment, the cells are mammalian cells in a mammal. In one embodiment, the coated particles, or freeze-dried or air-dried material, may be delivered to wounds, skin, tumor cells, mucosal tissue, retina and the like. In one embodiment, the particles are on a macrocarrier substrate. In one embodiment, the molecule comprises isolated nucleic acid, enzyme, antibacterial agent, antifungal agent, antiviral agent, or hormone. In one embodiment, the molecule is an antigen and so may be useful to vaccinate an animal. In one embodiment, the molecule is an enzyme and so may be useful for enzyme replacement therapy. In one embodiment, the molecule is a drug including biologics and so may be useful for other therapies including cancer therapy and wound healing.

In one embodiment, the invention provides a method to treat an animal having a disorder. The method includes providing an amount of a plurality of particles having a freeze-dried or air-dried coating with at least one molecule, wherein an amount of the molecule when administered to an animal is effective to inhibit or prevent at least one symptom associated with the disorder. The amount is biolistically delivered to an animal having the disorder. In one embodiment, the animal is a mammal, e.g., a human. In one embodiment, the disorder is cancer. In one embodiment, the amount is delivered to the epidermis of the animal.

If particles are employed in biolistic transformation, those particles may be formed of any material, including but not limited to gold, tungsten, silica, nickel, silver, platinum, palladium, titanium, iron, alumina, copper, metal alloys, calcium phosphate, emulsified wax, ceramics, carbon, chitosan, cellulose, lignine, chitin, starch, alginate, hyaluronan, dextran, cyclodextrins, dextran, arabinogalactan, pullulan, heparin, polystyrene, styrene, poly(vinylpyridine), polyvinyl alcohol, titanium oxide, cerium oxide, cadmium selenide or zinc sulfide, and may have a shape including but not limited to a sphere, a rod, a whisker, a cylinder, a tube, a cone, a prism, a polyhedron, with diameters or dimensions including from but not limited to, for a sphere 4 nm to 3 μm, for a rod 10×30 nm to 50×200 nm, for a whisker 5 nm to 5 μm, for a cylinder 5 nm to 5 μm, for a tube 5 nm to 5 μm, for a cone 4 nm to 3 μm, for a prism 4 nm to 3 μm, and for a polyhedron from 4 nm to 3 μm.

### BRIEF DESCRIPTION OF THE FIGURES

FIG. 1. Biolistic DNA delivery to plant tissues. A) Scheme of DNA precipitation onto 0.6 μm gold particles. A suspension of gold particles is mixed with a solution of plasmid DNA. A subsequent addition of calcium chloride and spermidine precipitates the plasmid DNA over the surfaces of the gold particles. These DNA coated projectiles are loaded in a macrocarrier (a plastic disk depicted) for bombardment. B) Scheme of a bombardment process. Pressurized helium is introduced in a chamber sealed with a plastic rupture disk. When the pressure in the chamber is higher than the resistance of the disk, it breaks releasing a helium blast that hits the macrocarrier where the DNA coated projectiles are held. The shock releases the projectiles that hit the plant tissue. Once the projectiles are inside the cells, the coated plasmid DNA is released and cell genome is transformed.

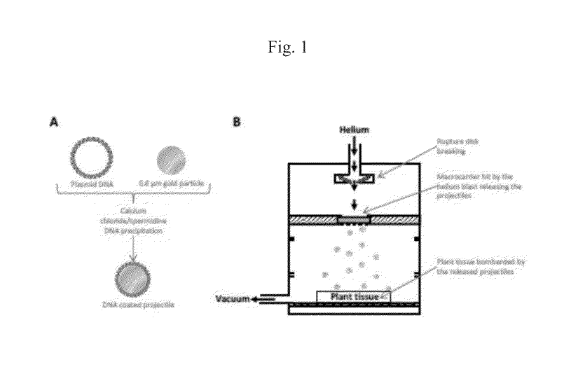

FIG. 2. Photomicrographs of plant cells bombarded with a GFP encoding plasmid or TRITC-BSA on gold particles. Left panels are black and white, upper right shows GFP expressing cells (green filter) and lower right shows TRITC-BSA delivery (red filter).

FIG. 3. Scheme of the lyophilization coating methodology and dye delivery to plant cells. A) A solution of the desired molecule, in this case the dye bromophenol blue, is mixed with a suspension of 0.6 μm gold microcarriers. This suspension is then placed in the center of a macrocarrier set and frozen in liquid nitrogen for several minutes. Subsequently, the frozen macrocarriers are lyophilized using a lyophilizer for 1 hour. Once the suspension is dried, it is ready to be used for plant tissue bombardment. B) Bright field image of an aliquot of bromophenol blue solution on top of onion epidermis tissue. The impermeable dye is not permeating inside the cells. C) Bromophenol blue delivery to onion epidermis cells upon bombardment with 0.6 μm gold lyophilized with the dye solution. Cells show different shades of the dye depending on the amount of microparticles-dye delivered.

FIG. 4. Delivery of DNA lyophilized onto 0.6 μm gold microcarriers. A) Bright field (left) and fluorescence (right) images of an onion epidermis tissue showing GFP expression 1 day after bombardment with the GFP expression plasmid pLMNC95 lyophilized onto 0.6 μm gold microparticles. B) Equivalent results in tobacco leaf tissue. C) Maize immature embryos showing blue foci after GUS staining assay. The embryos were bombarded with the uidA expressing plasmid pACH25 lyophilized onto the gold microcarriers. D) Bright field (left) and fluorescence (right) images of onion epidermis cells showing red fluorescence after being bombarded with a linear dsDNA for mCherry expression lyophilized onto the 0.6 μm gold microparticles.

FIG. 5. eGFP delivery to plant cells. Bright field (top) and fluorescence (bottom) images of a green fluorescent cell in which eGFP lyophilized onto 0.6 μm gold was delivered after bombardment.

FIG. 6. TRITC-BSA protein delivery to plant cells. A) Macrocarrier with a liquid suspension of TRITC-BSA protein and 0.6 μm gold (left) and the same macrocarrier after the lyophilization process (right). B) Bright field (left) and fluorescence (right) images of TRITC-BSA and 0.6 μm gold microcarriers deposited on macrocarrier surface after lyophilization. C) TRITC-BSA protein delivery to maize immature embryo scutellum cells after bombardment. In the bright field image (left) dark dots corresponding to microprojectiles can be detected. Protein delivery can be observed in the red fluorescence channel (right). D) Bright field (left) and fluorescence (right) images of tobacco cells showing red fluorescence due to TRITC-BSA protein delivery after bombardment.

FIG. 7. Viability of cells after protein delivery. A) From top to bottom, bright field, red fluorescence and green fluorescence channel images of an onion epidermis cell showing red fluorescence due to TRITC-BSA delivery after bombardment. The same cell is alive as is showing green fluorescence due to the fluorescein diacetate vital staining. B) Graph representing the mean and standard deviation of dead cells in onion epidermis tissue not bombarded (Not Bomb.), bombarded with 0.6 μm gold microcarriers (0.6 μm) or bombarded with TRITC-BSA lyophilized onto the surface of 0.6 μm gold (TRITC-BSA 0.6 μm). The bars represent the mean of the dead cell found in 12 optical fields obtained with a 5× objective scattered evenly throughout 4 different bombarded tissues per treatment.

FIG. 8. Delivery of active enzymes to plant tissues. A) Bright field image of onion epidermis tissue bombarded with β-glucuronidase lyophilized onto 0.6 μm gold projectiles. Cells where the enzyme has been delivered show blue coloration after GUS histochemical staining with X-gluc. B) Fluorescence optical field images taken with a 5× objective of onion epidermis tissue stained with fluorescein diacetate. Samples were not bombarded (Not Bomb.), bombarded with 0.6 μm gold (0.6 μm) or with trypsin (Trypsin) or RNAse (RNAse) lyophilized onto the microprojectiles. Non-fluorescent cells are considered dead cells and were the ones counted for a quantitative measurement. C) Graph representing the mean and standard deviation of the number of dead cells found in optical fields scattered evenly on the surface of 4 onion epidermis samples for each of the 4 treatments tested.

FIG. 9. Co-delivery of plasmid DNA and protein to plant cells. A) From top to bottom, bright field, green and red fluorescence channel images of onion epidermis cells fluorescing in green due to GFP expression from the plasmid pLMNC95 and fluorescing in red due to TRITC-BSA protein delivery. Plasmid DNA and protein were lyophilized simultaneously onto 0.6 μm gold microcarriers. B) Graph representing the mean and standard deviation of the number of onion epidermis cells expressing GFP 1 day after bombardment. Microprojectiles were prepared following a CaCl2/Spermidine plasmid DNA precipitation (Precip.), lyophilization of plasmid DNA with 0.6 μm gold (0.6 μm+DNA) or lyophilization of plasmid DNA with 0.6 μm gold and TRITC-BSA protein. 4 samples were counted per treatment.

FIG. 10. Microscope images of macrocarriers with DS-RED2+0.6 μm gold suspensions A) or protein alone solution B) air-dried overnight. The protein is shiny and clumps of protein or protein/0.6 μm gold can be seen in the surface.

FIG. 11. A) Microscope images of cells after intracellular DS-RED2 protein delivery 30 minutes after bombardment with air-dried protein-0.6 μm gold suspension. B) Intracellular DS-RED2 protein delivery after bombardment with air dried DS-RED2+0.6 μm gold suspension. This mixture was more efficient than protein alone.

FIG. 12. A) Microscope images of onion epidermis cells showing blue coloration after intracellular delivery of β-glucuronidase and overnight incubation with X-gluc solution after being bombarded with A) the air-dried protein solution or B) the air-dried protein+0.6 μm gold suspension.

FIG. 13. A) Image of the macrocarrier with a liquid solution of DS-RED2 fluorescent protein. B) Bright field image (left) and red channel image (right) of an onion epidermis tissue after bombardment. In both protein alone and protein with 0.6 μm gold tissue damage could be seen. No intact red cells could be observed.

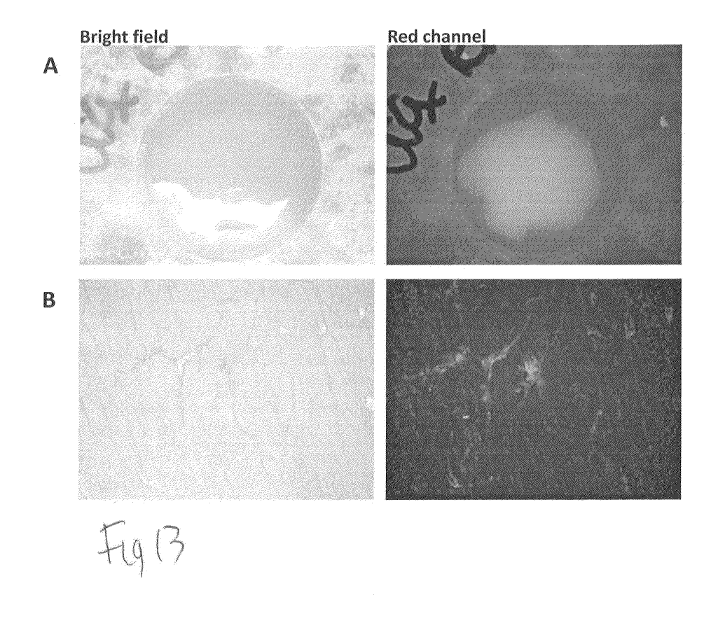

FIG. 14. A) Microscope image of onion tissue bombarded with the liquid solution of DS-RED2. No fluorescent cells were observed. Fluorescence was observed over the surface of the tissue. B) Bombardment with liquid β-glucuronidase. Tissue damage could be seen (right) and in some samples, areas of blue staining could be observed. The staining was over the surface of the cells, not inside the cells.

FIG. 15. Graph showing the number of fluorescent cells transiently expressing GFP after bombardment of onion epidermis tissue with 1 μg of plasmid DNA (pLMNC95) using the following procedures: CaCl₂/Spe: 0.6 μm gold coating with Calcium chloride/spermidine base protocol; freeze-dry: 0.6 μm gold coated freeze-drying the suspension onto the macrocarrier; freeze-dry DNA alone: freeze-dry the DNA onto the macrocarrier without 0.6 μm gold; air-dry: 0.6 μm gold coated air-drying the suspension onto the macrocarrier; and air dry DNA alone: freeze-dry the DNA onto the macrocarrier without 0.6 μm gold.

FIG. 16. A) Onion cell showing GFP expression after bombardment with DNA air-dried alone (no 0.6 μm gold). B) Onion cell showing GFP expression after bombardment with DNA freeze-dried alone (no 0.6 μm gold).

FIG. 17. A) Mouse ear pinna tissue before bombardment. B) Bright field image taken with a 40× microscope objective of an area of the pinna tissue after bombardment with air dried β-glucuronidase +0.6 μm gold after incubation in X-gluc solution overnight, where blue cells can be distinguished. These blue cells are the result of β-glucuronidase activity. C) Bright field images taken with a 63× microscope objective of one cell at two different depths (left and right images). In both depths of the same cell 0.6 μm gold particles can be detected (pointed with arrows).

FIG. 18. Onion epidermis cell showing blue coloration after incubation in X-gluc solution as a result of biolistic active β-glucuronidase enzyme delivery air-dried onto tungsten M5 (A) or M17 (B) particles. Onion epidermis tissue cells showing green fluorescence after expression of GFP expressing plasmid DNA pLMNC95 air dried onto tungsten M5 (C) or M17 (D) particles one day after bombardment.

FIG. 19. Scanning electron microscope images of 0.6 μm gold particles layout onto the macrocarrier (left: overall image; right: close up) after DNA coating with CaCl₂/spermidine (A), air-dry in Tris/NaCl buffer (B), air-dry in O-glucuronidase protein solution in Tris/NaCl buffer (C) and lyophilized in O-glucuronidase protein solution in Tris/NaCl buffer.

### DETAILED DESCRIPTION

### Definitions

The term “amino acid,” comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide). Other suitable amino and carboxy protecting groups are known to those skilled in the art (See for example, T. W. Greene, Protecting Groups In Organic Synthesis; Wiley: New York, 1981, and references cited therein).

The term “polypeptide” describes a sequence of at least 50 amino acids (e.g., as defined hereinabove) or peptidyl residues while a peptide describes a sequence of at least 2 and up to 50 amino acid residues. The sequence may be linear or cyclic. For example, a cyclic peptide can be prepared or may result from the formation of disulfide bridges between two cysteine residues in a sequence. A polypeptide can be linked to other molecules through the carboxy terminus, the amino terminus, or through any other convenient point of attachment, such as, for example, through the sulfur of a cysteine. In one embodiment of the invention a polypeptide comprises about 50 to about 300 amino acids. In another embodiment a peptide has about 5 to about 25 amino acids Peptide and polypeptide derivatives can be prepared as disclosed in U.S. Pat. Nos. 4,612,302; 4,853,371; and 4,684,620, or as described in the Examples hereinbelow. Polypeptide sequences specifically recited herein are written with the amino terminus on the left and the carboxy terminus on the right.

The term “nucleic acid”, “polynucleic acid” or “polynucleic acid segment” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991; Ohtsuka et al., 1985; Rossolini et al., 1994). An “oligonucleotide” typically includes 30 or fewer nucleotides.

As used herein, the terms “isolated and/or purified” refer to in vitro preparation, isolation and/or purification of a nucleic acid or protein (polypeptide or peptide) so that it is not associated with in vivo substances, or is substantially purified from in vitro substances. Thus, with respect to an “isolated nucleic acid molecule”, which includes a polynucleotide of genomic, cDNA, or synthetic origin or some combination thereof, or an “isolated polypeptide or peptide”, the “isolated nucleic acid molecule” or “isolated polypeptide or peptide” (1) is not associated with all or a portion of cell based molecules with which the “isolated nucleic acid molecule” or “isolated polypeptide or peptide” is found in nature, (2) is operably linked to a molecule which it is not linked to in nature, or (3) does not occur in nature as part of a larger sequence. An isolated nucleic acid molecule means a polymeric form of nucleotides of at least 10 bases in length, either ribonucleotides or deoxynucleotides or a modified form of either type of nucleotide. The term includes single and double stranded forms of DNA. The term “oligonucleotide” referred to herein includes naturally occurring, and modified nucleotides linked together by naturally occurring, and non-naturally occurring oligonucleotide linkages. Oligonucleotides are a polynucleotide subset with 200 bases or fewer in length. In one embodiment, oligonucleotides are 10 to 60 bases in length including 12, 13, 14, 15, 16, 17, 18, 19, or 20 to 40 bases in length. Oligonucleotides may be usually single or double stranded. Oligonucleotides can be either sense or antisense oligonucleotides. The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotides linkages such as phosphorothioate, phosphorodithioate, phosphoroselenoate, phosphorodiselenoate, phosphoroanilothioate, phosphoraniladate, phosphoroamidate, and the like.

The term “complexed” refers to binding of a molecule to a different molecule, typically through means other than covalent bonding. Such binding can take to form of, e.g., ionic or electrostatic interactions, or other attractive forces. For instance, DNA may be complexed with a protein.

### Exemplary Molecules and Compositions for Delivery

Many molecules are unable to cross the membrane barrier of cells without the assistance of transport systems. For example, hydrophilic molecules are generally unable to cross the membrane barrier of cells without transport mechanisms due to the hydrophobic nature of the lipid bilayer, e.g., proteins are generally unable to cross the membrane barrier of cells without the assistance of protein transport systems. This challenge has led to the development of protein delivery systems using materials including polymers, carbon nanotubes and mesoporous silica nanoparticles. There are a few examples of protein delivery methodologies to cells such as plant cells, such as microinjection and cell penetrating peptides. While these methodologies could be used to introduce model proteins into plant cells, they have major disadvantages including the requirement of skillful handling of cell materials or lack of protection of the introduced protein during the process.

Delivery of bioactive, e.g., proteins, or co-delivery of bioactive agents, such as protein and DNA, to plant cells has great biological significance. Thus, with respect to delivery of protein and nucleic acid, in addition to the potential of enhancing genetic transformation and gene targeting in plants, researchers could assess loss or gain of function of different post-translationally modified forms of a protein, and protein interactions with other biomolecules. Also, direct delivery and release of proteins in plant cells could facilitate the understanding of cellular machinery or signal pathways more effectively. For example, this would allow for a greater understanding of protein functions in host cells where protein production pathways are impaired, or analyzing cellular regulatory functions through delivery of antibodies.

The biolistic method, or gene-gun method, is based on the bombardment of living organisms, tissues or cells using projectiles. Nanoparticle mediated delivery of bioactive (biogenic) molecules to plant cells, such as double or single stranded DNA and small interfering RNA, and delivery and release of chemical substances such as phenanthrene and plant growth regulators, have been reported. However, biolistic methods to deliver molecules in an effective amount to, for instance, intact plant cells, depend on the density of the delivery vehicle and the loading capacity of the vehicle.

The projectiles for plant biolistics usually are between 0.4 and 1.5 μm in diameter and are made of tungsten or gold. The biolistic method has been used mainly to deliver nucleic acid sequences like plasmid DNA to cells in order to create genetically modified organisms. Usually, these nucleic acids are physically precipitated on the projectile surface following a calcium chloride-spermidine based precipitation protocol (Klein et al., 1987; Sanford et al., 1993). This method is broadly used in plant transformation experiments. Plant researchers use homemade gene-guns or commercially available ones like PDS-1000/He from Bio-Rad. In recent years, other research fields like human and animal medicine are using gene-guns to deliver, for example, DNA vaccines (Haynes, 2004; de Andrés et al., 2009).

Table 1 summarizes delivery systems that are primarily used for plant transformation and includes the present method.

TABLE 1

| | | Delivery | Substances | |
|---|---|---|---|---|
| System | Description | methodology | delivered | References |
| Freeze dry | Use sublimation to | Biolistic method | Chemicals, | Described here |
| method | coat chemicals or | | DNA, | |
| | biomolecules over | | proteins | |
| | particles. | | | |
| Diolistic | Use evaporation or | Biolistic method | Chemicals | (Gan et al., 2000; |
| | precipitation to | | | Shestopalov et |
| | coat dyes over | | | al., 2002; |
| | particles. | | | Lichtman et al., |
| | | | | 2005; Bothwell et |
| | | | | al., 2006; O'Brien |
| | | | | and Lummis, |
| | | | | 2006; Roizenblatt |
| | | | | et al., 2006; |
| | | | | O'Brien and |
| | | | | Lummis, 2007; |
| | | | | Coelho et al., |
| | | | | 2008; Gan et al., |
| | | | | 2009) |
| Modified | The surface of a | Biolistic method | DNA, | (Wu et al., |
| gold particle | gold particle was | | proteins | 2011b; Wu et al., |
| | modified to attach | | | 2011a) |
| | transposome, a | | | |
| | mix of an enzyme | | | |
| | and a DNA | | | |
| | sequence | | | |
| Nanoparticles - | Load different | Biolistic method | Chemicals, | (Grichko et al., |
| biolistic | types of | | DNA, | 2006; Torney et |
| | nanoparticles with | | proteins | al., 2007; Martin- |
| | the desired | | | Ortigosa et al., |
| | molecule. | | | 2012a; Martin- |
| | Nanoparticles will | | | Ortigosa et al., |
| | be used for | | | 2012b) |
| | bombardment. | | | |
| Nanoparticles - | Load different | Incubation | Chemicals, | (Pasupathy et al., |
| uptake | types of | | DNA | 2008; Wild and |
| | nanoparticles with | | | Jones, 2009; |
| | the desired | | | Silva et al., 2010) |
| | molecule. | | | |
| | Incubate the living | | | |
| | tissue with the | | | |
| | loaded | | | |
| | nanoparticle | | | |
| | suspension. | | | |
| Cell | Use the properties | Incubation | Chemicals, | (Chang et al., |
| penetrating | of these short | | DNA, | 2005; Wang et |
| peptides | peptides to cross | | proteins | al., 2006; Chang |
| | the membrane | | | et al., 2007; |
| | barrier to drag | | | Chugh and |
| | with them the | | | Eudes, 2007; |
| | desired molecule. | | | Chugh and |
| | There are | | | Eudes, 2008a; |
| | commercially | | | Chugh and |
| | available kits. | | | Eudes, 2008b; |
| | | | | Chugh et al., |
| | | | | 2009; |
| | | | | Eggenberger et |
| | | | | al., 2009; Lu et |
| | | | | al., 2010; |
| | | | | Eggenberger et |
| | | | | al., 2011; Qi et |
| | | | | al., 2011) |
| Microinjection | Use microneedles | Injection | Chemicals, | (Staiger et al., |
| | to deliver a | | DNA, | 1994; Wymer et |
| | molecule solution | | proteins | al., 2001) |
| Electroporation | Use | Electroporation | DNA, | (Hayashi and |
| | electroporation to | | proteins, | Kamiya, 2009) |
| | form pores in cells | | chemicals | |
| | to introduce | | | |
| | molecules | | | |

The present invention provides for freeze-dried or air-dried solutions of molecules, e.g., dyes, proteins and nucleic acids, coated onto particles, for biolistic delivery regardless of the presence or absence of a naturally occurring uptake or transport mechanism for the molecule, and may be used to deliver agents to any type of cell, including mammalian cells, e.g., ovine, porcine, equine, bovine, feline, canine or primates, such as humans, and plant cells.

The molecules for delivery include, but are not limited to, genes, nutrients (vitamins, etc.), and biocidal or pesticidal agents (e.g., insecticides or herbicides). For example, the term includes but is not limited to antibacterial agents, antifungal agents, antiviral agents, polypeptides, hormones, enzymes, antibodies, and RNA or DNA molecules of any suitable length, or any combination thereof. For instance, the RNA or DNA molecules may encode herbicide resistance, drought tolerance, a polypeptide associated with enhanced nutritional value, and the like.

Exemplary molecules for delivery to cells, including plant cells, include but are not limited to polypeptides, and/or polynucleotides (DNA or RNA) encoding a screenable marker, a polypeptide that can enhance or stimulate cell growth, an enzyme such as a recombinase, an integrase, a site-specific recombinase, a DNA topoisomerase, an endonuclease, a transposase, a restriction enzyme, a DNA polymerase, a DNA ligase, and the like, a transcription factor, a repressor, a DNA binding protein, a DNA repair protein a cell cycle protein, a RNA binding protein, RNase, a RNA-dependent RNA polymerase, ribosomal proteins, methyltransferase enzymes, hydroxylase enzymes, histone modifying enzymes, chromatin modifying enzymes, and the like.

In some examples the agent comprises a polynucleotide or polypeptide that stimulates cell growth. The agent employed in compositions for delivery to cells may provide a means for positive selection of recipient target cells, increased transformation efficiency, increased plastid transformation efficiency, increased gene targeting or combinations thereof. Genes that enhance or stimulate cell growth include genes involved in transcriptional regulation, homeotic gene regulation, stem cell maintenance and proliferation, cell cycle regulation, cell division, and/or cell differentiation.

The agent may be an antigen from any pathogen including any virus, bacteria, parasite or fungi that generates a pathological condition in an animal, e.g., one useful for a vaccine. The virus can be, for example, a herpesvirus, an influenza virus, a orthomyxovirus, a rhinovirus, a picornavirus, an adenovirus, a paramyxovirus, a coronavirus, a rhabdovirus, a togavirus, a flavivirus, a bunyavirus, a rubella virus, a reovirus, a measles virus, a hepadna virus, a filovirus, or a retrovirus (including a human immunodeficiency virus; including all clades of HIV-1 and HIV-2 and modifications thereof). The bacteria can be, for example, a mycobacterium (e.g., M. tuberculosis, which causes tuberculosis or M. leprae, which causes leprosy), a spirochete, a rickettsia, a chlamydia, or a mycoplasma. The parasite can be, for example, a parasite that causes malaria, and the fungus can be, for example, a yeast or mold. In one embodiment, the antigen is a glycoprotein. In one embodiment, the antigen is a viral capsid protein. In one embodiment, the antigen is a nonstructural protein, e.g., a protein that is not a viral polymerase. For example, the antigen may be any of the M, E or C proteins of West Nile virus, any of the N protein, P protein, M protein, F protein or glycoprotein of a paramyxovirus, or the capsid or a nonstructural protein of an astrovirus. In one embodiment, the antigen may be a toxin protein or a modified toxin protein, e.g., from Clostridium botulinum.

In one embodiment, the antigen may be a Meningococcus protein, a Streptococcus protein, a pneumococcus protein, a Neisseria protein or capsular polysaccharide, Hemophilus influenza protein, a Plasmodium falciparum protein, a mycobacterial protein, a protein from Bacillus anthracis, a protein from Corynebacerium diptheriae, a Bordetella pertussis protein, a protein or capsular polysaccharide from Salmonella typhi, a protein from Vibrio cholera, a HIV protein, a West Nile virus protein, a polio virus protein, a hepatitis B virus protein, a hepatitis C virus protein, an influenza virus protein, a respiratory syncytial virus protein or a dengue virus protein.

Pharmaceutical compositions of the present invention, suitable for inoculation comprise one or more agents such as antigens, e.g., isolated nucleic acid encoding one or more proteins thereof, optionally prepared (prior to freeze drying or air drying) using sterile aqueous or non-aqueous solutions, suspensions, and emulsions. The compositions can further comprise auxiliary agents or excipients, as known in the art. The composition of the invention is generally presented in the form of individual doses (unit doses). For instance, vaccines generally may contain about 0.1 to 200 mg, e.g., 30 to 100 μg, of protein into their composition.

When a composition of the present invention is used for administration to an individual, it can further comprise salts, buffers, adjuvants, or other substances which are desirable for improving the efficacy of the composition. For vaccines, adjuvants, substances which can augment a specific immune response, can be used. Normally, the adjuvant and the composition are mixed prior to presentation to the immune system, or presented separately, but into the same site of the organism being immunized.

A pharmaceutical composition according to the present invention may further or additionally comprise at least one chemotherapeutic compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-α, interferon-β, interferon-γ, tumor necrosis factor-alpha, thio semicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The administration of the composition (or the antisera that it elicits) may be for either a “prophylactic” or “therapeutic” purpose. In one embodiment, when provided prophylactically, the compositions of the invention which are vaccines are provided before any symptom or clinical sign of a pathogen infection becomes manifest. The prophylactic administration of the composition serves to prevent or attenuate any subsequent infection. In one embodiment, when provided prophylactically, a composition of the invention, is provided before any symptom or clinical sign of a disease becomes manifest. The prophylactic administration of the composition serves to prevent or attenuate one or more symptoms or clinical signs associated with the disease.

In one embodiment, when provided therapeutically, a vaccine is provided upon the detection of a symptom or clinical sign of actual infection by a pathogen. The therapeutic administration of the composition serves to attenuate any actual infection. In one embodiment, when provided therapeutically, a composition is provided upon the detection of a symptom or clinical sign of the disease. The therapeutic administration of the compound(s) serves to attenuate a symptom or clinical sign of that disease.

Thus, a vaccine composition of the present invention may be provided either before the onset of infection (so as to prevent or attenuate an anticipated infection) or after the initiation of an actual infection. Similarly, the composition may be provided before any symptom or clinical sign of a disorder or disease is manifested or after one or more symptoms are detected.

A composition is said to be “pharmacologically acceptable” if its administration can be tolerated by a recipient. Such an agent is said to be administered in a “therapeutically effective amount” if the amount administered is physiologically significant. A composition of the present invention is physiologically significant if its presence results in a detectable change in the physiology of a recipient, e.g., enhances at least one primary or secondary humoral or cellular immune response against at least one pathogen.

The “protection” provided need not be absolute, e.g., the infection need not be totally prevented or eradicated, if there is a statistically significant improvement compared with a control population. Protection may be limited to mitigating the severity or rapidity of onset of symptoms or clinical signs of the infection.

A typical regimen for preventing, suppressing, or treating a pathogen related pathology, comprises administration of an effective amount of a vaccine composition, administered as a single treatment, or repeated as enhancing or booster dosages, over a period up to and including between one week and about 24 months, or any range or value therein.

According to the present invention, an “effective amount” of a composition is one that is sufficient to achieve a desired effect. It is understood that the effective dosage may be dependent upon the species, age, sex, health, and weight of the recipient, kind of concurrent treatment, if any, frequency of treatment, and the nature of the effect wanted.

The invention will be further described by the following non-limiting examples.

### Example I

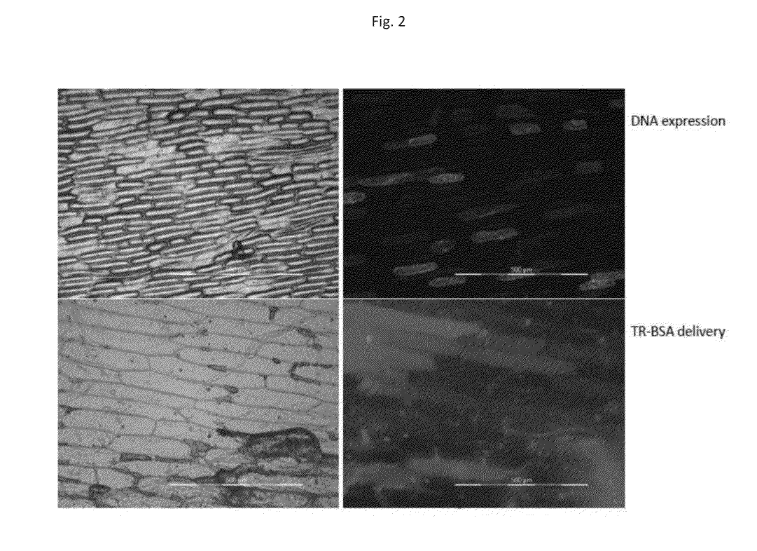

FIG. 2 shows results obtained using 0.6 μm gold particles coated with DNA or protein using a freeze dry process. 2 μL of 0.6 μm gold particle stock was mixed with 1 μg of GFP plasmid pLMNC95 or 2 μL of a 400 ng/μL, TRITC-BSA solution. The mixture was poured on a macrocarrier, frozen in liquid nitrogen and freeze-dry for 1 hour prior to onion epidermis tissue bombardment.

200-800 cells were found to express GFP per sample, which is comparable to CaCl₂/spermidine precipitation. TRITC-BSA was also successfully delivered to cells.

### Example II

### Methods

### Plant Materials

Onion epidermis tissue was obtained from the scale leaves of white onion bulbs. Rectangular (3×2.5 cm) pieces were peeled right before the bombardment and placed in solid agar media (0.5 mM of 2-(N-morpholino)ethanesulfonic acid (MES), 15 g L⁻¹ Bacto agar (BD), pH 5.7) with the peeled side upwards. For quantitative measurements, epidermis pieces of the same scale leaf were distributed among treatments. Tobacco leaves (Nicotiana tabacum var. Petite Havana) were obtained from 3-6 week old in vitro grown plants on MS media (MS medium (Murashige and Skoog, 1962), 2% sucrose, 2.5 g L⁻¹ gelrite, pH 5.7). Leaves were cut right before bombardment and put with the adaxial surface up on agar media. Hill maize immature embryos were obtained from immature ears provided by the Center for Plant Transformation—Iowa State University. Immature embryos (1-2 mm long) were cultured as previously described (Frame et al., 2000).

### Microparticle-Molecule Lyophilization

The saturated solutions of chemical or biomolecules used were done as follows (data per shot): for bromophenol blue (Sigma) delivery 10 μL of a 100 μg 1 μL⁻¹ mg solution were used. For plasmid DNA delivery, 2 μL of a 500 ng μL⁻¹ solution of the plasmid pLMNC95 (Luke Mankin and Thompson, 2001) were used for GFP expression. For mCherry expression linear dsDNA, the plasmid ER-rk (Nelson et al., 2007) was digested with the restriction enzymes SacI-HindIII. The digestion was electrophoresed in a 0.8% agarose gel and the 1885 bp band was cut and purified using a gel DNA extraction kit (IBI Scientific). For the delivery, 14 μL of a 2.8 ng μL⁻¹ solution of the purified linear dsDNA were used. Plasmids pLMNC95 and ER-rk were obtained from the Arabidopsis Biological Resource Center. For maize embryo bombardment, 1 μg of plasmid pACH25 (Christensen and Quail, 1996) was used. For delivery of eGFP (Biovision) 2.5 μL of a 1 μg μL⁻¹ were used. TRITC-BSA, β-glucuronidase and trypsin were dissolved in 250 mM NaCl, 15 mM Tris pH 8 buffer. For TRITC-BSA (Invitrogen), β-glucuronidase (Sigma), trypsin (Sigma) and RNAse A (Sigma) delivery, 10 μL of a 25 μg μL⁻¹ solution, 5.5 μL of a 50 μg uL⁻¹ solution, 4 μL of a 50 μg μL⁻¹ solution and 4 μL of a 10 μg μL⁻¹ solution respectively were used.

The solutions above described were mixed by pipetting with 2 μL of a 30 μg μL⁻¹ solution in water of 0.6 μm gold microcarriers (Cat. 165-2262, Bio-Rad) per shot. The mix was loaded and evenly distributed over the inner circle (the central part that gets hit by the helium blast) of the macrocarrier set (a plastic macrocarrier already placed into the macrocarrier holder). The loaded macrocarrier set was frozen in liquid nitrogen for 5-10 minutes and freeze-dried using a lyophilizer (Freezone 2.5 from Labconco) for at least 1 hour. Bombardments were performed right after the lyophilization process.

### Biolistic Method

For plant tissue bombardment the PDS-1000/He gene gun (Bio-Rad) was used according to the general settings described previously (Frame et al., 2000). All the gene gun supplies used are from Bio-Rad. For onion epidermis tissue, 1100 psi rupture discs and a 6 cm target distance were used. For tobacco leaf and maize immature embryos, 650 psi and 6 cm target distance bombardment conditions were used.

### X-Gluc Histochemical Staining

The procedure followed has been described previously (Jefferson, 1987). The X-gluc substrate was from Biosynth. Right after bombardment, bombarded tissues were soaked in the X-gluc solution overnight at 37° C.

### Fluorescein Diacetate Staining

Onion epidermis tissues were submerged in 10 mL of MS liquid media to which 100 μL of a 5 mg mL⁻¹ of fluorescein diacetate (Alfa Aesar) solution in acetone were added. The samples were incubated for 2-5 minutes and directly observed under the green channel filter of the fluorescence microscope. For quantitative measurements, 12 images (10.5×8 mm) were taken with the 5× microscope objective scattered every 0.5 cm (4 columns×3 rows). The total amount of dead cells found on each image was counted.

### Microscopy

Bright field and fluorescence images were taken using a Zeiss Axio star plus microscope. The objectives used were A-plan 5×/0.12 and 10×/0.25. For the green channel fluorescence images a GFP BP filter (Chroma Technology Corp.) was used (λex=470 nm, beam splitter=495 nm and λem=525 nm). For the red channel, a Texas Red filter (Chroma Technology Corp.) was used (λex=560 nm, beam splitter=595 nm and λem=645 nm). Images were taken with the ProgRes Capture Pro 2.6 software and the ProgRes C3 digital camera (Jenoptik). False magenta color of red channel images was edited using Adobe Photoshop.

### Results

Delivery of a Chemical Lyophilized onto Microparticles

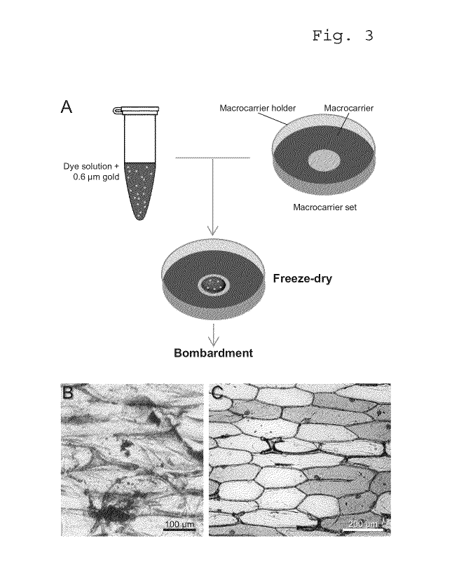

The gene gun used to deliver different molecules to plant cells is the PDS1000/He gun from BioRad, a gene gun routinely used in plant transformation experiments. Usually, gold microparticles or microcarriers (0.6-1 μm diameter) are coated with plasmid DNA following a CaCl₂/Spermidine precipitation protocol (Frame et al., 2000). These projectiles are suspended in an ethanol solution and poured over the surface of a “macrocarrier set”, composed of a plastic macrocarrier arranged in a macrocarrier holder (FIG. 3A). When ethanol evaporates, the projectiles remain stuck to the macrocarrier surface and are ready to be bombarded.

Lyophilization or freeze drying consists in the removal of a solvent from a frozen sample by sublimation and is routinely used to preserve labile molecules like proteins (Tang and Pikal, 2004). Hypothetically, any molecule could be coated onto the surface of the microcarriers after lyophilizing a solution containing the molecule of interest and microparticles, e.g., 0.6 μm gold particles (FIG. 3A). For a better delivery of these coated microprojectiles the lyophilization process was done on the macrocarrier set.

The dye bromophenol blue was used to test the ability of the system to deliver a chemical. As a control, a saturated solution of the dye (10 μL of a 100 μg μL⁻¹ mg) was deposited with a pipette on top of onion epidermis tissue. The dye did not permeate and remained outside the cells (FIG. 3B). For the bromophenol blue delivery, the saturated solution was mixed with 0.6 μm gold microcarriers and an aliquot was deposited over a macrocarrier set. This macrocarrier set was frozen in liquid nitrogen for 5-10 minutes and then lyophilized for an hour using a lyophilizer. This macrocarrier set was subsequently used to bombard onion epidermis tissue. As soon as 30 minutes after bombardment, several cells were showing bluish-purple coloration, indicating intracellular delivery of bromophenol blue throughout the cytoplasm (FIG. 3C). The amount of dye delivered to each cell was different, and as a consequence, different shades of purple could be observed in the bombarded tissue (FIG. 3C).

Delivery of DNA Lyophilized onto Microparticles

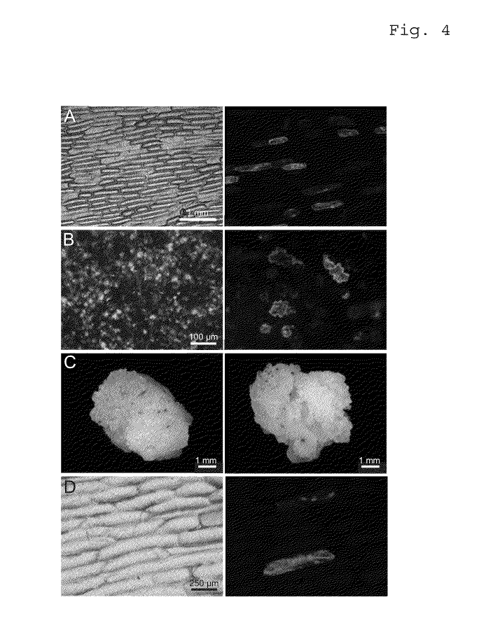

Delivery of plasmid DNA to plant cells is a routine technique used in plant biotechnology. As mentioned, plasmid DNA is usually coated onto microcarriers following a calcium chloride and spermidine based DNA precipitation protocol. In theory, lyophilizing plasmid DNA onto the surface of microcarriers could also be used as a coating protocol. The plasmid pLMNC95 (Luke Mankin and Thompson, 2001) for GFP expression was mixed with 0.6 μm gold particles and, as in the case of bromophenol blue, a lyophilization process was followed to coat the microparticles over a macrocarrier set. These macrocarriers were used to bombard different plant tissues. One day after bombardment, several GFP fluorescent cells could be observed in onion epidermis cells (FIG. 4A) and to tobacco leaf cells (FIG. 4B). The delivery of uidA gene containing plasmid DNA pACH25 (Christensen and Quail, 1996) to maize immature embryos was also tested. After lyophilizing the mixed suspension of pACH25 and 0.6 μm gold microparticles over a macrocarrier set, maize HiII immature embryos were bombarded. The embryos showed multiple blue foci after X-gluc histochemical staining (FIG. 3C). These results confirm the use of lyophilization as a method to coat the projectiles for the delivery of plasmid DNA to different plant tissues.

Furthermore, coating 0.6 μm gold with linear double stranded DNA (dsDNA) was also tested. The 1.8 Kb linear DNA cassette for mCherry expression was obtained after digestion of plasmid ER-rk (Nelson et al., 2007). Following the same protocol, linear dsDNA was mixed with 0.6 μm gold microparticles and the suspension lyophilized in a macrocarrier set. Intracellular delivery of mCherry dsDNA was confirmed when red fluorescent cells were observed 1 day after bombardment in onion epidermis tissue (FIG. 4D).

Proteolistics: Delivery of Proteins Lyophilized onto Microparticles

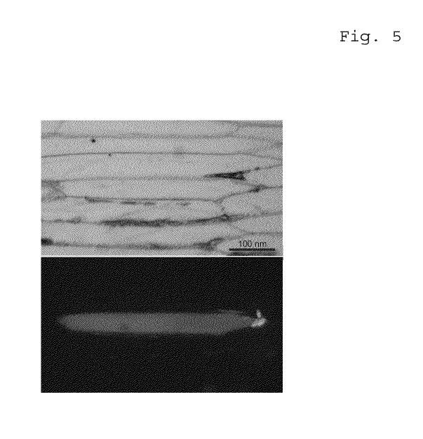

Protein delivery can be considered a difficult challenge because proteins could be subjected to denaturation during the delivery process. To prove the delivery of an intact protein through the lyophilization coating method eGFP was chosen. This protein will not fluoresce if denatured (Ward and Bokman, 1982). As a control, to test if the protein could be diffused into plant cells, 10 μL of a 100 ng/μL eGFP solution were incubated for 30 minutes on the surface of intact onion epidermis tissue or bombarded with bare 0.6 μm gold microparticles. No intracellular eGFP detection could be observed (data not shown). Lyophilization of eGFP onto 0.6 μm gold microcarriers was done using 2.5 μL of 1 μg μL⁻¹ solution of the protein and 60 μg of microcarriers per shot. Green fluorescent cells could be observed 30 minutes after bombardment (FIG. 5) indicating successful intracellular delivery of intact eGFP.

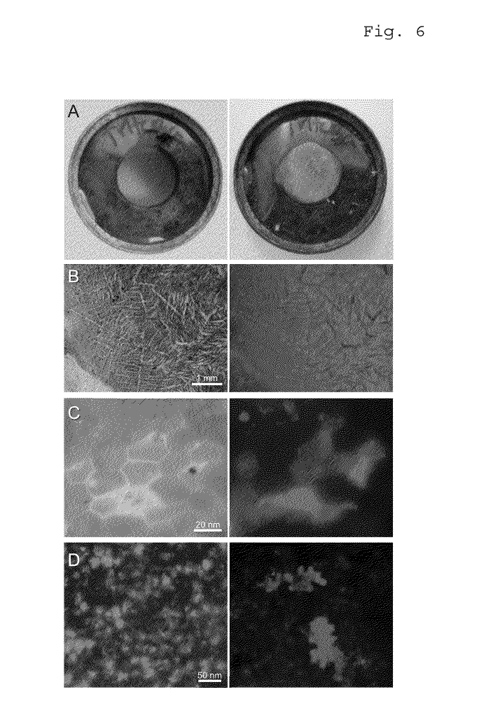

To corroborate this protein delivery methodology, tetramethylrhodamine isothiocyanate labeled bovine serum albumin (TRITC-BSA) was used. Per shot, 10 μL of a 25 μg μL⁻¹ solution were mixed with 60 μg of 0.6 μm gold microcarriers. This suspension was distributed over the center of a macrocarrier set (FIG. 6A left), frozen in liquid nitrogen and lyophilized for an hour (FIG. 6A right). The lyophilized mix of protein and microparticles could be observed attached to the macrocarrier, and it showed red fluorescence (FIG. 6B). Maize Hi II immature embryo scutella (FIG. 6C) and tobacco leaves (FIG. 6D) were bombarded with the protein coated microprojectiles. TRITC-BSA delivery could be observed in both tissues after bombardment. Cells showed different intensities of red fluorescence according to the amount of protein delivered, what was related to the number of particles that reached each cell. This can be observed in detail in FIG. 6C where the amount of black dots (0.6 μm gold particles) per cell is related to the fluorescence intensity or TRITC-BSA amount delivered.

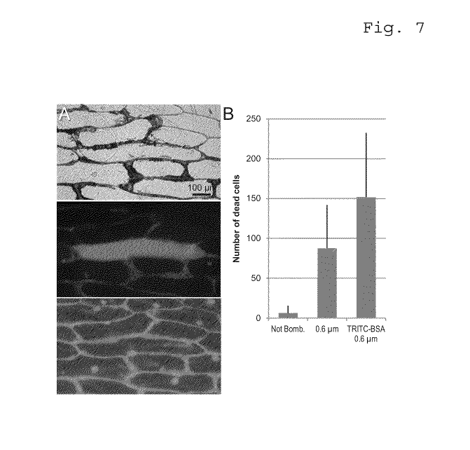

The high concentration of protein used per shot (250 μg per shot) and the lyophilization process could cause cell death by: (1) physical damage of the potential clumps produced after lyophilizing protein and microparticles and (2) excess of intracellular protein delivery reaching toxic levels. To assess cellular death, onion epidermis tissues were stained with fluorescein diacetate, which stains in fluorescent green the living cells, while the dead ones remain dark (FIG. 7A). Onion epidermis cells not bombarded, bombarded only with 0.6 μm gold projectiles or with TRITC-BSA coated projectiles were stained 1 day after bombardment with fluorescein diacetate. In general, most of the cells showing TRITC-BSA delivery were alive, even the ones showing a high protein delivery (FIG. 7A). The number of dead cells found in the samples bombarded with TRITC-BSA coated projectiles was higher than the ones bombarded without the protein (FIG. 7B). Due to the high variability in the number of dead cells in each treatment these differences were not significant.

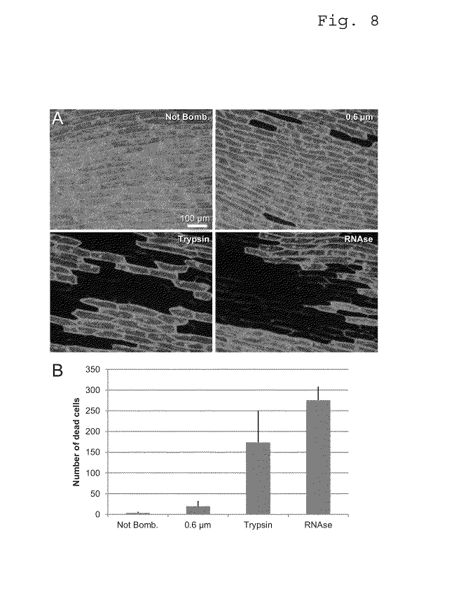

The next step was to assess the delivery of enzymes and confirm activity in cells. β-glucuronidase (275 μg per shot) was lyophilized onto 0.6 μm gold projectiles and onion epidermis tissue was bombarded. Right after bombardment samples were incubated in an X-gluc solution for histochemical staining. One day after bombardment several blue cells could be observed in the bombarded samples (FIG. 8A) showing that active β-glucuronidase was delivered.

As another example of active enzyme delivery, trypsin and RNAse A were chosen. These enzymes, if active, should cause cell death when delivered in enough amounts to digest intracellular protein or RNA content. The number of dead cells was measured using fluorescein diacetate staining in samples not bombarded, bombarded only with 0.6 μm gold or with 200 μg of trypsin or 40 μg of RNAse per shot. An optical field obtained with a microscope 5× objective for each treatment is presented in FIG. 8B. In samples not bombarded, the majority of the cells were alive. In samples bombarded with 0.6 μm gold, some dead cells were present probably due to physical damage caused by the projectiles. The samples bombarded with the projectiles coated with the enzymes showed vast areas of dead cells, an indirect measurement of the activity of the enzymes. The graph (FIG. 8C) shows the quantitative measurement of this damage. The samples bombarded with trypsin or RNAse showed large amounts of dead cells comparing to the controls without the enzymes.

### Co-Delivery of Plasmid DNA and Protein

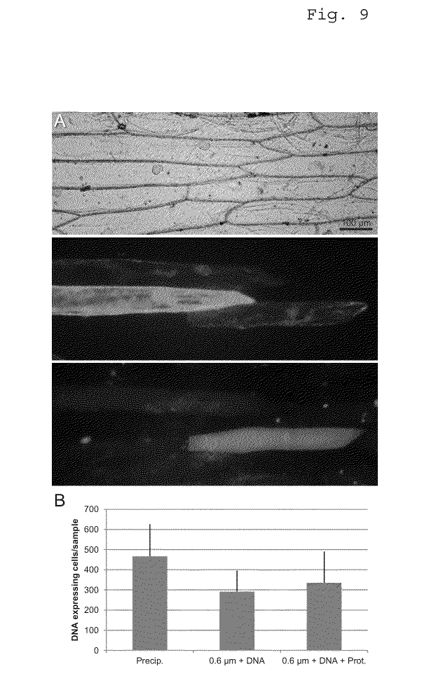

In theory, any compatible mixed solution of chemicals and biomolecules could be lyophilized onto the projectiles. To assess the co-delivery of two biomolecules, GFP expression plasmid pLMNC95 and TRITC-BSA protein were lyophilized onto 0.6 μm gold. One day after bombardment, the co-delivery of both biomolecules could be observed in the same cells (FIG. 9A) in onion epidermis tissue. Cells fluorescing simultaneously in green (due to plasmid DNA expression) and in red (due to TRITC-BSA protein delivery) could be observed as a consequence of the co-delivery (FIG. 9A). Since the protein/plasmid DNA co-delivery could affect DNA expression, a quantitative measurement of DNA expression was performed. As shown in FIG. 7B, the amount of cells expressing DNA after a traditional plasmid DNA precipitation using calcium chloride and spermidine (Precip.) was higher than the ones obtained after lyophilizing the plasmid (0.6 μm+DNA) for the same amount of DNA (1 μg). Nevertheless, there were no differences between the number of cells expressing GFP after lyophilizing DNA or DNA with TRITC-BSA (FIG. 9B).

### Discussion

Lyophilizing protein, chemicals and other biomolecules along with microparticles offers a projectile coating method for the efficient intracellular delivery of these molecules through the biolistic method. For more than two decades the main purpose of the biolistic method has been the delivery of DNA. This methodology has been broadly used by biologists to manipulate cell genomes for basic and applied research. Recently, new molecule delivery methodologies like cell penetrating peptides or nanotechnology mediated delivery are being developed. The main purpose of these methodologies is the delivery of other biomolecules, in particular, the delivery of proteins (Chugh et al., 2009; Ravichandran, 2009; Lu et al., 2010; Martin-Ortigosa et al., 2012a). These methodologies require the design and synthesis of the vehicles (peptides or nanoparticles) that will allow the delivery of the molecules what could be complicated. Additionally, in the case of nanoparticle mediated protein delivery, nanoparticle surfaces can interact with the proteins affecting the structure or activity upon adsorption (Kane and Stroock, 2007) or remain stuck in the nanoparticle pores (Martin-Ortigosa et al., 2012a).

On the contrary, lyophilization based protein coating of microprojectiles, “Proteolistics,” is a simple and straight forward method that offers an easy, cost effective and quick alternative for the delivery of any protein or molecule combination that can be delivered by the biolistic method to the required target tissue. In this work, we have shown the biolistic delivery to different plant tissues of chemical dyes like bromophenol blue (FIG. 3), active enzymes like β-glucuronidase, trypsin and RNAse A (FIG. 8) and the co-delivery of two biomolecules like plasmid DNA and TRITC-BSA protein (FIG. 9). Furthermore, a gene gun and a lyophilizer are equipment that can be easily found in any research facility.

In order to expand biolistic methodology uses, dyes have been delivered to different tissues (Gan et al., 2000; Bothwell et al., 2006; Roizenblatt et al., 2006; O'Brien and Lummis, 2007) by the diolistic approach. In this case, a dye is precipitated over the surface of a microparticle after evaporation of the dye solvent. Then, these dye covered microparticles are used as projectiles to deliver the chemical to cells upon bombardment. In this research, lyophilization has been used for the successful delivery of bromophenol blue, and it also has been applied to other labile biomolecules like proteins (FIGS. 5, 6 and 8) or biomolecule combinations (FIG. 9). Lyophilization is widely used to preserve labile molecules (Tang and Pikal, 2004), so this process not only allows quick projectile coating, it also prevents biomolecule from degradation.

In the experiments described above, saturated solutions of molecules have been used to be mixed with the 0.6 μm gold microparticles. While in some embodiments the delivery of a high concentration of a macromolecule such as a protein may be desired, because this methodology does not offer a controlled release, there may be a concentration that could be toxic for a cell.

Plant genome editing or enzyme assisted plant transformation are hot topics in plant sciences since a precise engineering of the genome is highly desirable. For instance, Wu and colleagues have developed methodologies in which transposon and transposase complexes are attached using different approaches to gold microparticles to improve plant transformation (Wu et al., 2011a; Wu et al., 2011b). As described herein, the simple combination of protein and DNA lyophilized with the microparticles can effectively deliver both molecules to plant tissues upon bombardment, without modifying the enzyme, the DNA sequence or the gold microparticle. This result indicates that proteolistics could be routinely used for these enzyme assisted gene editing purposes.

This methodology could also be applied for the biolistic delivery of molecules to other organisms (Obregón-Barboza et al., 2007), or even be useful for biomedical purposes (Davidson et al., 2000; Yager et al., 2009; Davtyan et al., 2012). This coating methodology may be applied to any type of projectile and to any type of compatible molecule combination.

### Example III

### Delivery of Proteins Air-Dried onto Gold Projectiles

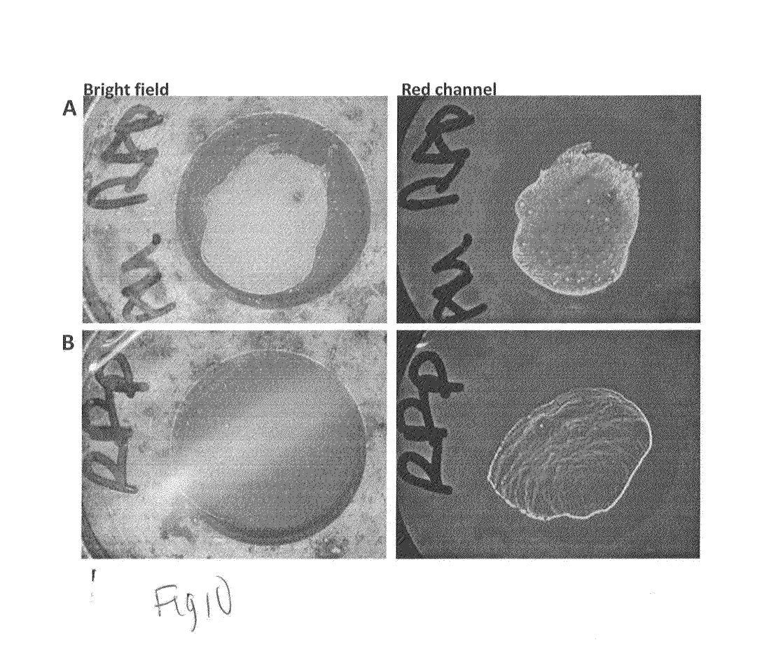

Previously, protein and 0.6 μm gold projectiles were mixed and poured on top of a macrocarrier of the gene gun, frozen in liquid nitrogen, and lyophilized or freeze-dried. The same experiment was repeated by pouring the protein/0.6 μm gold mixture over a macrocarrier and leaving it to dry on top of a bench for 1 hour or overnight, termed “air-dried” (FIG. 10A). The experiment was done with two different proteins, β-glucuronidase and DS-RED2. Onion epidermis tissues were bombarded using these macrocarriers.

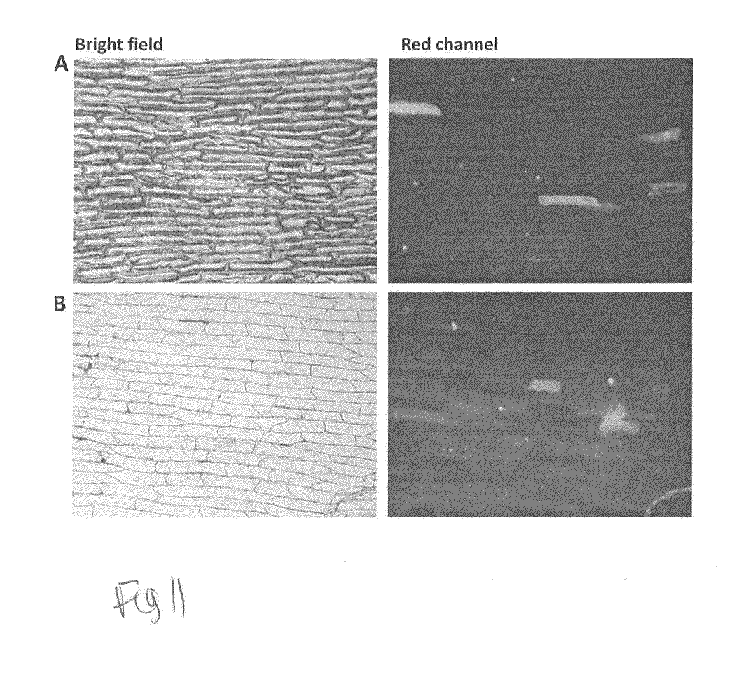

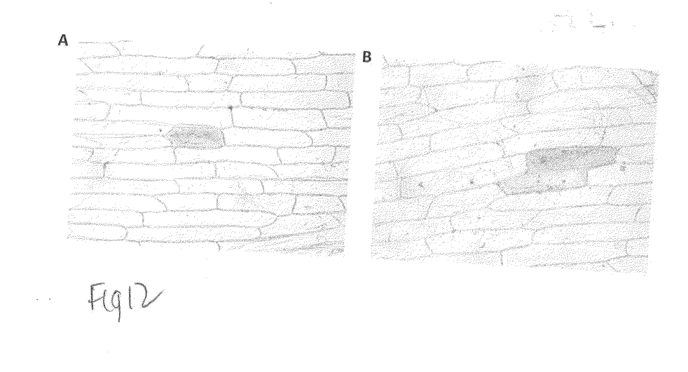

The results showed that, in both cases, protein delivery was achieved (FIGS. 11B and 12B). Although certain proteins may be subject to degradation, β-glucuronidase and DS-RED2 proteins were not substantially degraded when subjected to air drying.

Delivery of Proteins Air-Dried Directly onto Macrocarriers (without Gold Projectiles).

An experiment was conducted using β-glucuronidase and DS-RED2, air-dried directly onto the macrocarriers without 0.6 μm gold projectiles (FIG. 10B). After onion epidermis tissue bombardment, protein delivery also could be detected (FIGS. 11A and 12A) showing that projectiles are not necessary for delivery, even though protein delivery using projectiles is more efficient.

Bombardment of Tissues with Liquid Solutions of the Proteins.

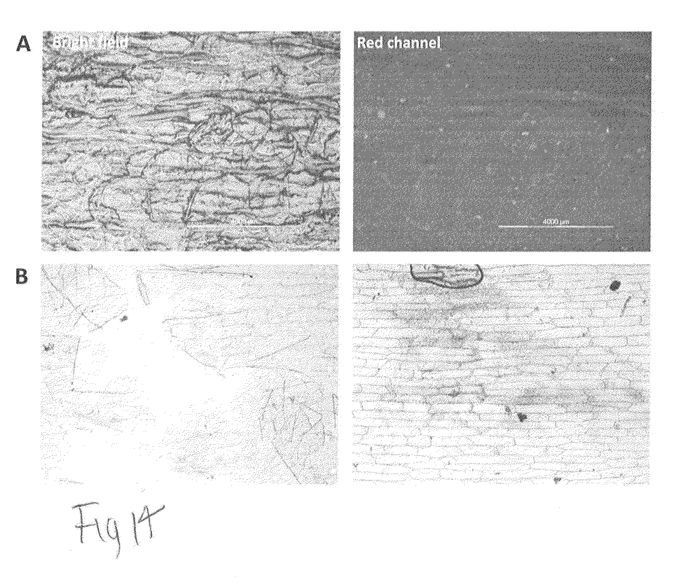

As a control, β-glucuronidase and DS-RED2 protein solutions were poured over the macrocarrier and onion epidermis cell tissues were bombarded. Tissue damage was caused by the liquid propelling (FIG. 14) and no protein delivery was observed. However, the use of lesser volumes, less harsh gene-gun conditions or other liquid propelling methodologies may allow for protein delivery with less tissue damage.

Delivery of DNA Air-Dried onto 0.6 μm Gold Projectiles.

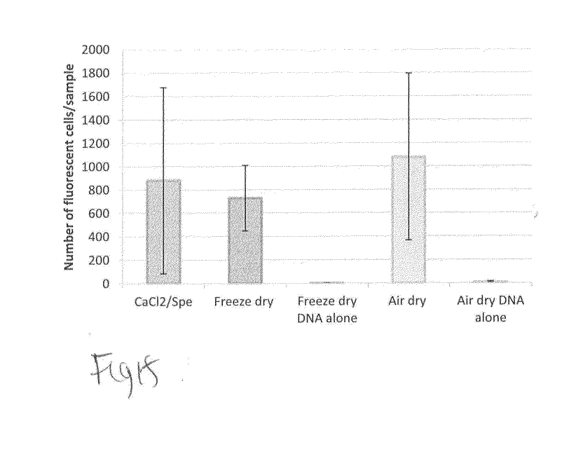

An experiment was conducted to compare DNA coating based on CaCl₂/spermidine precipitation, freeze-drying or air-drying, and 0.6 μm gold particles. As seen in FIG. 15, the number of cells transiently expressing GFP after plasmid DNA delivery is similar in the three methods.

Delivery of DNA Air-Dried or Freeze-Dried Directly onto Macrocarriers (without Gold Projectiles).

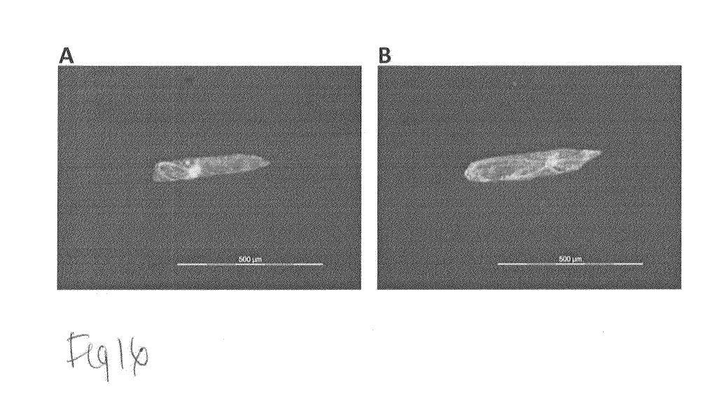

A DNA containing solution was freeze-dried or air-dried (without 0.6 μm gold projectiles) directly onto macrocarriers. Onion epidermis tissues were bombarded and 1 to 20 fluorescent cells could be observed (FIG. 15 and FIG. 16). Even though the number of cells that were transfected is much lower than when using 0.6 μm gold, protein can be delivered without the use of projectiles.

### Example IV

### Delivery of Active β-Glucuronidase to Mouse Ear Pinna Tissue Cells

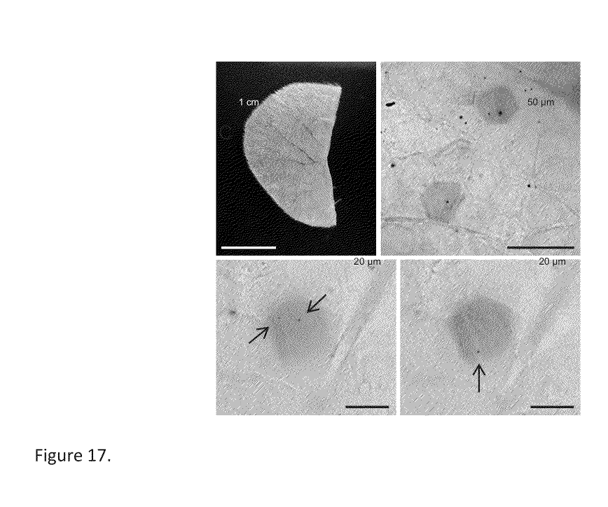

A mixture of 90 μg of 0.6 μm gold and 200 μg of β-glucuronidase per shot were air-dried onto the macrocarrier set for 2 hours. Ear pinnas of 8 week old CD-1 mice were dissected right after animals were euthanized. The tissues were disposed with the inner part of the ear upwards on an agar medium plate (FIG. 17A) and were immediately bombarded using the PDS-1000/He gene-gun at 650 psi or 1100 psi, 6 cm target distance and 28 mmHg vacuum. Samples were then soaked in X-gluc solution and incubated at 37° C. overnight. Sixteen hours later samples were examined under the microscope. In both conditions (650 and 1100 psi) several blue cells were localized in the tissues (FIG. 17B). Multiple gold particles (detected as dark dots) could be detected in different depths of the same cell (FIG. 17C). The localization of these blue cells in the bombarded tissue is the result of active β-glucuronidase protein delivery.

### Example V

### Intracellular Delivery of Active β-Glucuronidase or Plasmid DNA to Plant Tissues Air Dried onto Tungsten Particles

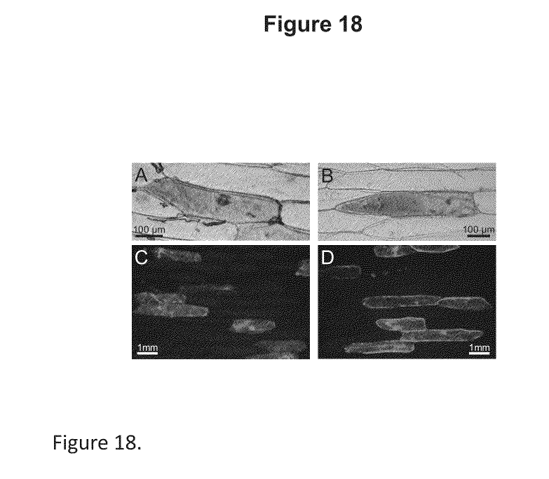

To prove the technique in other type of particles, tungsten particles were used. M5 (0.4 μm in size) and M17 (1.1 μm in size) tungsten particles from Bio-Rad were used. 100 μg of each type of particle and 200 μg of β-glucuronidase enzyme or 1 μg of GFP expressing plasmid pLMNC95 were used per shot. The suspensions were air dried for 2 hours onto the macrocarrier-set. Onion epidermis tissues were bombarded at 1100 psi/6 cm. Samples bombarded with β-glucuronidase were incubated overnight at 37° C. in X-gluc solution as previously described. After this incubation period, several cells were showing blue coloration due to active β-glucuronidase enzyme delivery air-dried onto M5 tungsten (FIG. 18A) or M17 tungsten (FIG. 18B).

Green fluorescent cells could be observed 1 day after bombardment in the onion epidermis tissues bombarded with the GFP expressing plasmid pLMNC95 air dried onto M5 tungsten (FIG. 18C) or M17 tungsten (FIG. 18D).

### Example VI

### Scanning Electron Microscope Images of Protein:Gold Particle Mixture Air-Dried or Lyophilized onto Macrocarriers

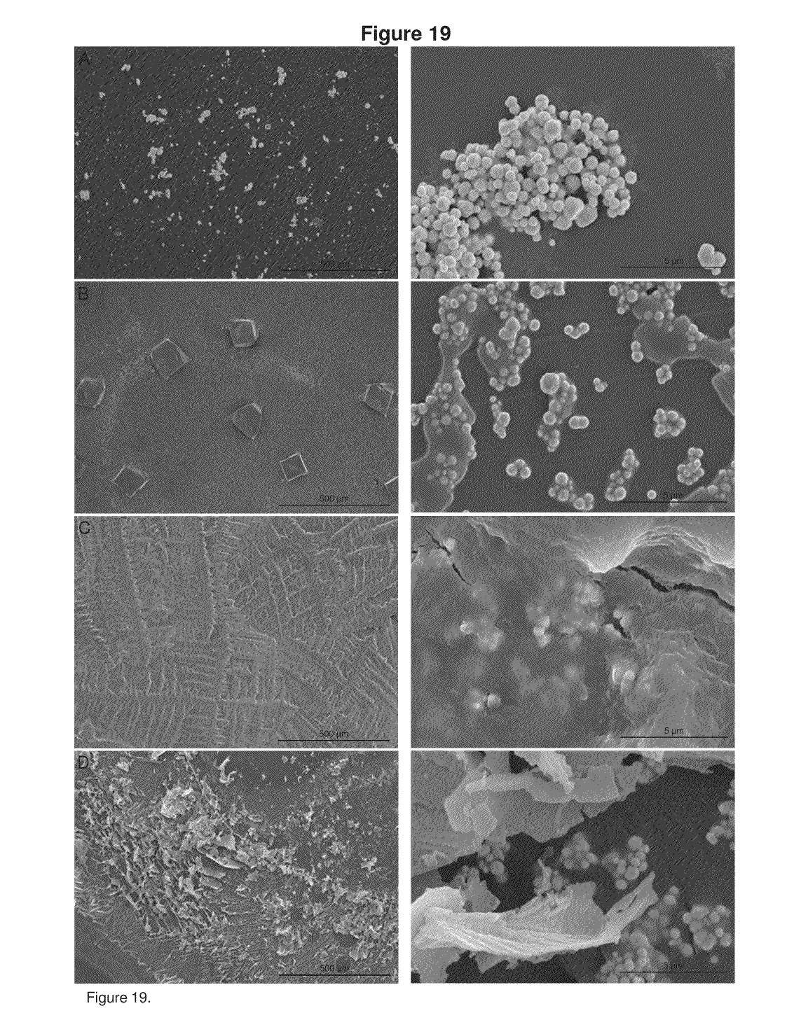

Sixty μg of gold particles (0.6 μm in diameter) were coated with 1 μg of DNA following the calcium chloride-spermidine protocol (FIG. 19A), air-dried with a Tris/NaCl buffer without any protein (FIG. 19B), air-dried (FIG. 19C) or lyophilized (FIG. 19D) with 250 μg of β-glucuronidase (50 μg/μL solution) in Tris/NaCl buffer. The macrocarriers holding these different treatments were mounted onto aluminum stubs and lightly sputter coated with palladium/gold alloy target on a Denton Desk II Sputter coater (Denton Vacuum, LLC, Moorestown, N.J.). Images were taken using a JEOL (Japan Electron Optics Laboratories, Peabody, Mass.) 5800LV scanning electron microscope at 10 kV.

As seen in FIG. 19, different treatments resulted in different layouts of the particles onto the macrocarrier. The DNA coated particles were found in clusters of different sizes scattered throughout the macrocarrier (FIG. 19A). In the case of particles mixed with Tris/NaCl buffer and air dried for 1 hour, particles were embedded in a solid matrix formed by the saline solution (FIG. 19B). When 250 μg of the protein β-glucuronidase were added to the gold particles and air dried onto the macrocarrier, the protein-saline solution dried forming a fern-like solid matrix embedding the particles (FIG. 19C). When this mix was lyophilized, the physical changes occurring during the sublimation of the buffer lead to laminated tridimensional structures (FIG. 19D). It is speculated that the solid structures shown in FIGS. 19C and D are broken during the bombardment and reach the cell releasing the crystallized protein:particle mix.

### REFERENCES

Bothwell et al., Plant J., 46:327 (2006).
Chang et al., New Phytol., 174:46 (2007).
Chang et al., Plant Cell Physiol., 46:482 (2005).
Christensen and Quail, Transgenic Res., 5:213 (1996).
Chugh and Eudes, Biochimica et Biophysica Acta (BBA)—Biomembranes, 1768:419 (2007).
Chugh and Eudes, FEBS J., 275:2403 (2008b).
Chugh and Eudes, J. Pept. Sci., 14:477 (2008a).
Chugh et al., Plant Cell Rep., 28:801 (2009).
Coelho et al., Planta, 227:1037 (2008).
Davidson et al., Wound Repair and Regeneration, 8:452 (2000).
Davis et al., PNAS, 96:8687 (1999).
Davtyan et al., Neurodegenerative Dis., 10:261 (2012).
de Andrés et al., Vaccine, 27:4591 (2009).
Eggenberger et al., Chem. BioChem., 10:2504 (2009).
Eggenberger, et al., Chem. BioChem., 12:132 (2011).
Frame et al., In Vitro Cell Dev-Pl., 36:21 (2000).
Gan et al., Ballistic Delivery of Dyes for Structural and Functional Studies of the Nervous System, Cold Spring Harb. Protoc. 2009, pdb.prot5202-(2009).
Gan et al., Neuron., 27:219 (2000).
Grichko et al., NanoBioTechnology, 2:37 (2006).
Hayashi and Kamiya, In: Methods in Cell Biology—Stephen, M. K., and Gregory,
J. P., eds.: Academic Press, 107 (2009).
Haynes, Exp. Op. Biol. Ther., 4:889 (2004).
Jefferson, Plant Mol. Biol. Rep., 5:387 (1987).
Kane and Stroock, Biotechnology Progress, 23:316 (2007).
Klein et al., Nature, 327:70 (1987).
Lichtman et al., Method for labeling individual cells United States: Washington University (2005).
Lu et al., J. Agric. Food Chem., 58:2288 (2010).
Luke Mankin and Thompson, Plant Mol. Biol. Rep., 19:13 (2001).
Martin-Ortigosa et al., Adv. Funct. Mat., 22:3576 (2012a).
Martin-Ortigosa et al., Small, 8:413 (2012b).
Murashige and Skoog, Physiologia Plantarum, 15:473 (1962).
Naqvi et al., J. Mat. Chem., 22:3500 (2012).
Nelson et al., Plant J., 51:1126 (2007).
Obregón-Barboza et al., J. Virology, 140:124 (2007).
O'Brien and Lummis, Nat. Protocols, 1:1517 (2006).
O'Brien and Lummis, Trends Biotechnol., 25:530 (2007).
Pasupathy et al., Biotech. J., 3:1078 (2008).
Qi et al., Mol. Plant-Microbe Inter., 24:25 (2011).
Ravichandran, NanoBioTechnology, 5:17 (2009).
Roizenblatt et al., J. Neuro. Meth., 153:154 (2006).
Sanford et al., Methods Enzymol., 217:483 (1993).
Shestopalov et al., Exp. Eye Res. 74:639 (2002).
Silva et al., BMC Plant Biol., 10:291 (2010).
Staiger et al., Curr. Biol., 4:215 (1994).
Tang and Pikal, Pharm. Res., 21:191 (2004).
Torney et al., Nat. Nanotech. 2:295 (2007).
Vijayakumar et al., Adv. Funct. Mat., 20:2416 (2010).
Wang et al., Biochem. Biophys. Res. Commun., 346:758 (2006).
Wang et al., J. Nanosc. and Nanotech., 11:2208 (2011).
Ward and Bokman, Biochem., 21:4535 (1982).
Wild and Jones, Environ. Sci. Technol., 43:5290 (2009).
Wu et al., Plant J., 68:186 (2011b).
Wu et al., Plant Mol. Biol., 1-11 (2011a).
Wymer et al., Planta, 212:692 (2001).
Yager et al., Exp. Rev. Vaccines, 8:1205 (2009).
   All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. A plurality of particles for biolistics of about 0.3 μm to about 1.2 μm in diameter having a freeze-dried or air-dried coating of at least one isolated molecule.

2. The plurality of particles of claim 1 wherein the at least one the isolated molecule includes isolated protein.

3. The plurality of particles of claim 1 wherein the isolated molecule includes isolated nucleic acid and isolated protein.

4. The plurality of particles of claim 1 wherein the isolated molecule includes a drug.

5. A method to deliver particles for biolistic delivery of at least one molecule comprising:
a) providing a substrate having a solution with a mixture of a plurality of particles and at least one isolated molecule;
b) freeze-drying or air-drying the solution in or on the substrate to provide a preparation of particles coated with the at least one molecule; and
c) biolistically delivering the plurality to eukaryotic cells in an amount effective to deliver the at least one molecule into the cells, wherein if the cells are not plant cells, the particles are about 0.3 μm to about 1.2 μm in diameter.

6. The method of claim 5 wherein the particles are about 0.3 μm to about 1.2 μm in diameter.

7. The method of claim 5 wherein the molecule is not isolated ribonucleic acid.

8. The method of claim 5 wherein the cells are plant cells.

9. The method of claim 5 wherein the at least one molecule is a protein or a peptide.

10. The method of claim 9 wherein the protein is a recombinase, an endonuclease or an enzyme that otherwise modifies nucleic acid.

11. The method of claim 10 wherein the molecule is a DNA ligase, polymerase, restriction enzyme, recombinase, such as Cre, FLP, R or Gin, or a nuclease such as a zinc finger nuclease or a transcription activator effector nuclease.

12. The method of claim 5 wherein the particles are coated with isolated nucleic acid and isolated protein.

13. The method of claim 5 wherein the cells are in a plant.

14. The method of claim 5 wherein the cells are in a mammal.

15. The method of claim 5 wherein the particles are on a macrocarrier.

16. The method of claim 5 wherein the particles are about 0.2 μm to about 2 μm in diameter.

17. The method of claim 5 wherein the at least one molecule comprises isolated nucleic acid, enzyme, antibacterial molecule, antifungal molecule, antiviral molecule, or hormone.

18. A method to vaccinate an animal, comprising:
a) providing a plurality of particles coated having freeze-dried or air-dried coating with at least one molecule, wherein the at least one molecule is an antigen; and
b) biolistically delivering the plurality to an animal in an amount effective to immunize the animal.

19. The method of claim 18 wherein the antigen is a protein or a peptide.

20. The method of claim 18 wherein the antigen is a viral antigen, a bacterial antigen, a virus a bacterium, or allergen.
